(19) United States
(12) Patent Application Publication
(10) Pub. No.: US 2007/0265702 A1
(43) Pub. Date: Nov. 15, 2007

(54) Percutaneous treatment for heart valves

(76) Inventor: Omar M. Lattouf, Atlanta, GA (US)

Correspondence Address: Edward J. Lynch;DUANE MORRIS LLP, One Market, Spear Tower, Suite 2000, San Francisco, CA, 94105, US

(21) Appl. No.: 11/698,643

(22) Filed: Jan. 25, 2007

Related U.S. Application Data
(60) Provisional application No. 60/763,098, filed on Jan. 27, 2006.

(51) Int. Cl. A61F 2/24

(52) U.S. Cl. 623/2.12

(57) ABSTRACT

The invention is directed to percutaneous transvascular therapeutic procedures, particularly for patients with congestive heart failure, and systems for such procedures. A system of the invention for a “Bow-tie” procedure has an elongated guide catheter, a leaf stabilizing device and a tissue grasping device for grasping the free edges of the patient's heart valve slidably disposed within the guide catheter. Preferably, an artificial cordae tendenae is provided if a natural cordae tendenae of the patient has been torn.

### CROSS-REFERENCE TO RELATED APPLICATIONS

This application relates to application Ser. No. 10/295,390, filed on Nov. 15, 2002 which is related to Provisional Application No. 60/340,062, filed Dec. 8, 2001, Provisional Application Ser. No. 60/365,918, filed Mar. 20, 2002, and Provisional Application Ser. No. 60/369,988, filed Apr. 4, 2002. The entire contents of these applications are incorporated herein by reference.

### BACKGROUND OF THE INVENTION

This invention is directed to therapeutic procedures for a patient's heart and to instruments and systems for such procedures. The invention is particularly suitable for treating a patient suffering from the symptoms of congestive heart failure (CHF), and particularly to those CHF patients exhibiting mitral valve regurgitation (MVR).

There are over five million patients in the United States suffering from CHF and there are more than seven hundred thousand new cases of CHF each year. For many of these patients medical therapy is not very successful.

With many CHF patients, their ventricular ejection fraction is reduced due to mitral valve regurgitation (MR) which may also result from dilated cardiomyopathy. The MR in turn can exacerbate the cardiomyopathy leading to a worsening of the MR. The MR can also be the result of torn cordae tendenae which extend from the valve leaflets to the papillary muscles, preventing complete closure of the valve.

Surgical procedures for mitral valve repair for MR typically involves installing a valve support ring at the base of valve. Recent advances in mitral valve repair include securing together the free edges of the mitral valve leaflets by sutures, staples and the like, commonly called “Bow-Tie” or “edge to edge” techniques. These procedures usually involve open heart surgery, including cardiopulmonary bypass and a sternotomy, although more recently suggestions have been made of performing these procedures with minimally invasive and percutaneous techniques which can reduce the morbidity of such procedures. Percutaneous procedures impose difficulties in instrument design because the instruments for such procedures must be long enough to extend from the entry location on the patient's leg to the interior of the patient's heart chamber, and they must have small enough profile and have sufficient flexibility for advancement through the patient's vasculature into the patient's heart chamber. Additionally, the instruments for such percutaneous procedures must also be able to accurately locate the operative distal ends of such instruments at a desired location within the chambers of the patient's beating heart and be strong enough to perform the required functions.

Techniques for Bow-Tie repair of mitral valves have been mentioned in the patent literature, but specific instruments for such techniques are not yet commercially available.

### SUMMARY OF THE INVENTION

This invention generally relates to percutaneous, transvascular therapeutic procedures, including valve repair, for patients with CHF and to the devices and systems suitable for use in such procedures. Specifically, one feature of the invention is directed to gaining access to the patient's heart valve, preferably from within the heart chamber. Such access may be gained through the patient's vasculature such as the femoral or brachial arteries or the subclavian vein. Such accessing can be effected through a previously positioned guide catheter which has a distal extremity that is shaped or shapeable to provide a desirable discharge orientation, such as toward the delivery site, for treatment instruments.

The guide catheter is configured to enable passage of instruments for the procedure to the treatment site. It may be provided with a sub-selective inner tubular member for proper discharge orientation within the patient's heart chamber toward the treatment site.

The instruments for performing the procedure are passed through the guide catheter with the proximal ends of these instruments extending out of the patient to allow the instruments to be manually or robotically manipulated to accurately position the operative ends of the instruments at the desired location within the heart chamber to perform the procedure and to operate the operative member(s) which may be provided on the distal ends of these instruments from outside the patient's body.

For “Bow-Tie” valve repair on a beating heart, the valve leaflets should be stabilized to facilitate grasping the leaflets with a suitable grasping device at a grasping location and then securing the free edges of the valve leaflets together by suitable connecting members such as one or more sutures, clips or staples or adhesive to form the “Bow-Tie” connection. A suitable stabilizing instrument, particularly for mitral or atrioventrical valve repair, is an elongated catheter having one or more expandable members on a distal location thereof, such as expandable arms or struts, or an inflatable balloon which can engage the surface, e.g. atrial surface, of the valve leaflets to stabilize and urge the valve leaflets toward a grasping location. The grasping member grasps and holds the free edges of the valve leaflets together from the opposite side of the valve so that the free edges can be secured together by a suitable connecting member or element. The elongated stabilizing instrument is advanced through the guide catheter into the patient's heart chamber defined in part by a ventricular wall until the distal extremity of the stabilizing instrument is advanced through the heart valve into the heart chamber beyond the heart valve, which in the case of the mitral valve is the left atrium. The expandable member(s) e.g. arms or struts or an inflatable balloon are expanded and then the stabilizing instrument is pulled proximally so the expandable member(s) engage the atrial side of the valve leaflets and move the valve leaflets into the grasping location within the ventricular chamber, e.g. left ventricle.

An elongated grasping device with at least a pair of grasping members such as jaws on the distal end thereof is advanced through the guide catheter until the distal end of the device extends out of the distal end of the guide catheter or a subselective tubular member thereof into the heart chamber. The grasping members or jaws of the grasping device are operated from the proximal end of the grasping device which extends out of the proximal end of the guide catheter which extends outside of the patient. The jaws of the grasping device are opened to receive the stabilized valve leaflets in the grasping location and then closed to grip the leaflets so that the free edges of the valve leaflets are placed into an operative position for the “Bow-Tie” repair. The free edges of the grasped valve leaflets may be joined or otherwise secured together by one or more suitable connecting elements. Once the free edges of the valve leaflets are secured together, the instruments for the procedure may be withdrawn through the guide catheter and then the guide catheter can be removed from the patient's ventricular system. The puncture wound provided for access into the patient's vasculature can be closed in a conventional manner, e.g. as in angioplasty/stent delivery procedures.

If there is cordae tendenae damage with the heart valve, particularly when there is severance of the cordae tendenae from the valve leaflet or the papillary muscle, repair of the valve leaflet, even by means of the “Bow-Tie” technique, may not prevent reshaping of the ventricular architecture which can reduce ventricular output. In that instance, it has been found that providing an artificial cordae tendenae such as a strand with one end secured to one or more of the free edges of the secured valve leaflets and another end secured to the heart wall, particularly in the same orientation as the natural cordae tendenae, will support the connected valve leaflets in more or less a normal manner to minimize ventricular deformation (e.g. dilated cardiomyopathy) which can lead to decreased output. One end of the strand may be secured to the connecting element securing the free edges of the valve leaflets or to the connected free edges themselves and the other end of the strand is secured to a location on the inner surface of the heart wall. The strand should be relatively inelastic or non-compliant to ensure an effective closed position of the leaflets. A suitable strand material is polytetrafluoroethylene (PTFE). Other suitable materials include other fluoropolymers, Nylon and polyethylene terephthalate. The pull on the valve leaflets by the strand of the artificial cordae secured thereto is in approximately the same orientation as the natural pull by the competent cordae tendenae. This provides for a better seal of the leaflets and thereby minimizes leakage through the valve.

The blood flow output from the CHF patient's heart due to the valve repair in accordance with the present invention is greatly increased, and leads to significant improvement in the physical well being, the life extension and the quality of life of the CHF patient. Moreover, due to the percutaneous transvascular delivery of instruments in this procedure, many of the CHF patient population, who are otherwise unsuitable for conventional surgical treatments, may be treated with the present procedures.

These and other advantages of the invention will become more apparent from the following detailed description and accompanying exemplary drawings.

### BRIEF DESCRIPTION OF THE DRAWINGS

FIG. 1 is a partial elevational view in section of a patient's left ventricle and left atrium illustrating an incompetent mitral valve with a torn cordae tendenae connected to one of the valve leaflets.

FIG. 2A is a transverse cross-sectional view illustrating an incompetent mitral valve in a closed condition during systole.

FIG. 2B is a transverse cross-sectional view illustrating the incompetent valve in an open condition during diastole.

FIGS. 3A and 3B are transverse cross-sectional views similar to those shown in FIGS. 2A and 2B but illustrating a competent mitral valve.

FIGS. 4A and 4B are transverse cross-sectional views similar to those shown in FIGS. 2A and 2B but illustrating an incompetent mitral valve with the valve leaflets thereof secured together in a “Bow-Tie” configuration.

FIG. 5 is an schematic perspective view of an assembly embodying features of the invention with a guide catheter having an operative proximal handle and with a stabilizing device and a grasping device within the inner lumen of the guide catheter.

FIG. 6 is an elevational view of a grasping device embodying features of the invention.

FIG. 7 is a transverse cross-sectional view of the grasping device shown in FIG. 5 taken along the lines 7-7.

FIG. 8 is a transverse cross-sectional view similar to that of FIG. 7 of an alternative grasping device with strands or wires for deflecting or shaping the distal end of the device.

FIG. 9 is an enlarged longitudinal cross-sectional view of the distal end of the grasping device with a valve leaflet connecting clip slidably disposed within the inner lumen of the grasping device.

FIGS. 10-12 are transverse cross-sectional views taken along the lines 10-10, 11-11 and 12-12 respectively of the grasping device shown in FIG. 9.

FIG. 13 is a transverse cross-sectional view taken along the lines 13-13 of the grasping device shown in FIG. 9 illustrating the pusher bar pushing the leaflet connecting clip along the guide way lumen of the grasping device.

FIG. 14 is an enlarged view of the distal end of the grasping device as shown in FIG. 10 with a leaflet connecting clip partially pressed into a connecting relationship with the free edges of the valve leaflets.

FIG. 15 is a transverse cross-sectional view taken along the lines 15-15 shown in FIG. 14 illustrating the clip partially connected to the valve leaflets.

FIG. 16 is an enlarged elevational view of the clip with an artificial cordae tendenae strand at the closed end of the clip.

FIG. 17 is an alternative clip construction which has an eyelet at the closed end thereof for securing the end of the artificial cordae tendenae.

FIG. 18 is an elevational view of a stabilizing device.

FIG. 19 schematically illustrates a patient's major arteries and delivery of a guide catheter through the patient's femoral artery and the patient's aortic valve and into the patient's left ventricle.

FIG. 20 illustrates shaping the distal portion of the guide catheter to orient the discharge port within the left ventricle towards the patient's mitral valve.

FIG. 21 illustrates advancing the stabilizing member out the discharge port in the grasping member.

FIG. 22 illustrates the expanded arms of the expandable member on the distal end of the stabilizing member engaging the upstream or atrial side of the valve leaflets and the jaws of the grasping member grasping the free edges of the valve leaflets.

FIG. 23 illustrates the leaflets grasped by the grasping member with the stabilizing member withdrawn into the guide catheter.

FIG. 24 illustrates the free edges of the leaflets secured together by a clip and an artificial cordae tendenae having one end secured to the clip and one end secured to the ventricular wall.

FIG. 25 illustrates the artificial cordae tendenae in a taut condition.

FIG. 26 illustrates the artificial cordae tendenae in a flaccid condition when blood flows through the valve into the ventricular chamber.

FIG. 27 illustrates a percutaneous, transvascular procedure wherein a guide catheter is introduced into the patient's right subclavian vein and advanced therein into the right atrium and through the atrial septum into the left atrium.

FIG. 28 illustrates advancement of the guide catheter through the incompetent mitral valve into the patient's left ventricle.

FIG. 29 illustrate engagement of the expanded the distal portion of the stabilizing member with the atrial surface of the valve leaflets and grasping the free edges thereof by the jaws of the grasping member.

FIG. 30 illustrates the mitral valve leaflets grasped by the grasping member with the stabilizing member withdrawn into the guide catheter.

FIG. 31 illustrates a strand of an artificial cordae tendenae secured at one end to the clip securing the valve leaflets and secured to the ventricular wall.

FIG. 32 illustrates the free edges of the leaflets secured together by a clip with an artificial cordae tendenae having one end secured to the clip and one end secured to the ventricular wall.

The drawings are schematic presentations and are not necessarily to scale.

### DETAILED DESCRIPTION OF EMBODIMENTS OF THE INVENTION

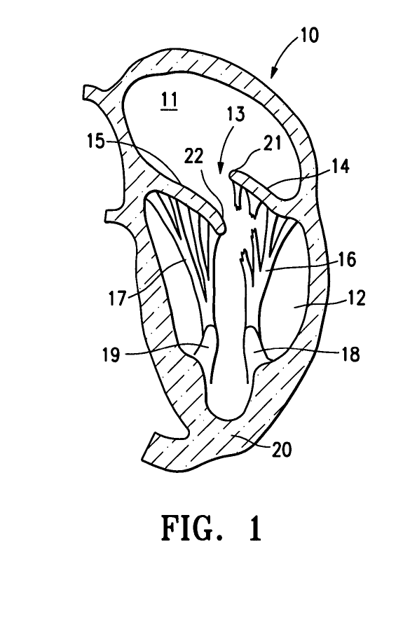

FIG. 1 illustrates the left side of a patient's heart 10 in partial cross-section schematically showing the patient's left atrium 11 and left ventricle 12 with a mitral valve 13 disposed between the left atrium and the left ventricle having a posterior valve leaflet 14 and an anterior leaflet 15. Each of the valve leaflets 14 and 15 have cordae tendenae 16 and 17 respectively which are connected to the leaflets and to papillary muscles 18 and 19 respectively within the left ventricle at the apex 20 of the heart. The posterior leaflet 14 of the mitral valve 13 is shown with its cordae tendenae 16 partially torn. The free edge 21 of the posterior leaflet 14 is uncontrolled due to the torn cordae tendenae 16 which makes the valve incompetent to close completely when the heart contracts during systole. The incompletely closed mitral valve 13 results in regurgitation of blood back through the valve into the atrium 11 during systole which in turn results in lowered blood output for the left ventricle 12. The anterior valve leaflet 15 is shown with its cordae tendenae 17 completely attached.

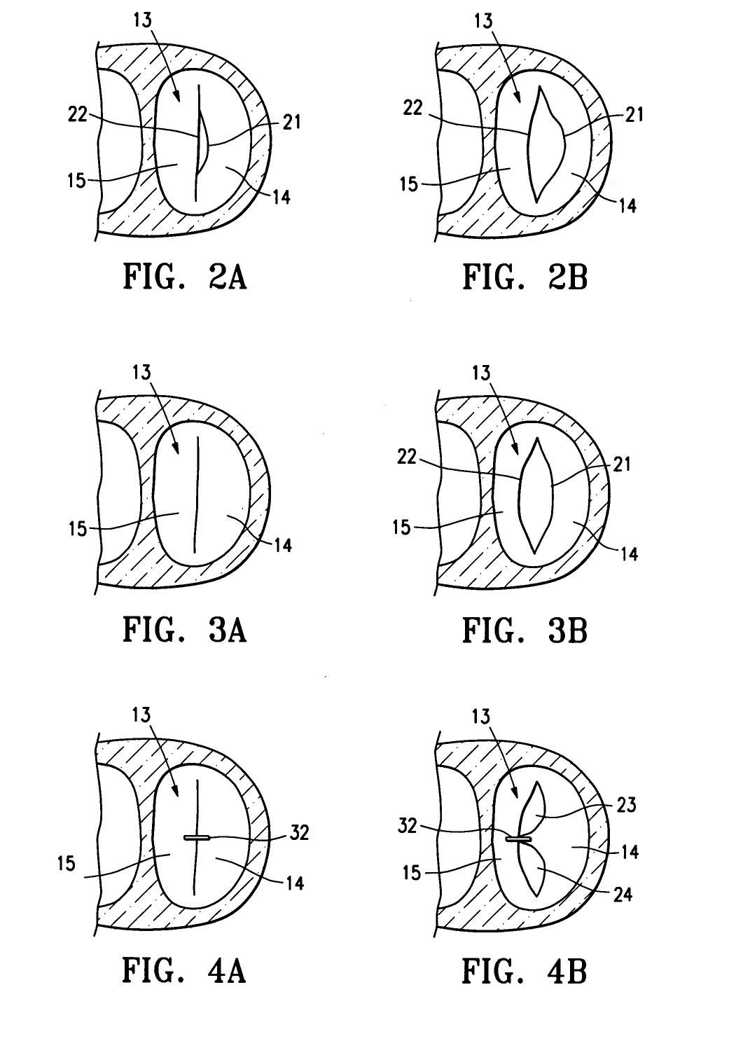

FIGS. 2A and 2B illustrate the closed and open condition respectively of an incompetent mitral valve 13 such as that shown in FIG. 1. The free edge 21 of posterior valve leaflet 14 is unable to close completely against the free edge 22 of anterior leaflet 15 due to the torn cordae tendenae as depicted in FIG. 1. A similar leaflet condition may occur due to dilated ventricular architecture, i.e. dilated cardiomyopathy, which is also characteristic of congestive heart failure.

FIG. 3A illustrates a healthy competent mitral valve 13 with valve leaflets 14 and 15 which are closed completely during systole to prevent regurgitation of blood through the valve. FIG. 3B illustrates the competent mitral valve shown in FIG. 2A in an opened condition during diastole to allow blood to flow from the left atrium to the left ventricle.

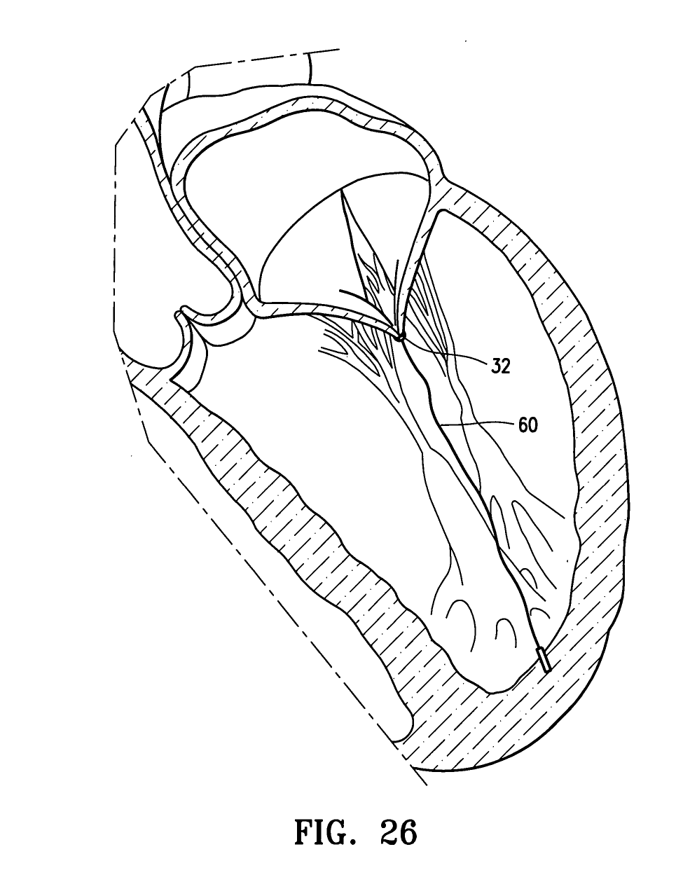

FIGS. 4A and 4B illustrate the closed and opened conditions of a mitral valve 13 in which the free edge 21 of posterior valve leaflet 14 and the free edge 22 of the anterior leaflet valve 15 are secured together in a “Bow-Tie” connection by a suitable clip, such as is shown in FIG. 26. During systole, when the heart contracts, the clip holds the free edges 21 and 22 of the valve leaflets together to minimize blood regurgitation through the valve. However, during diastole, when the heart muscle relaxes and the blood pressure within the left ventricle 12 is reduced, the mitral valve 13 opens up much like a competent valve but with two openings 23 and 24 between the valve leaflets 14 and 15. The interference with blood flow through the two openings 23 and 24 of a repaired mitral valve with a Bow-Tie connection between the leaflets is minimal during diastole compared to the flow with a single opening for a competent mitral valve.

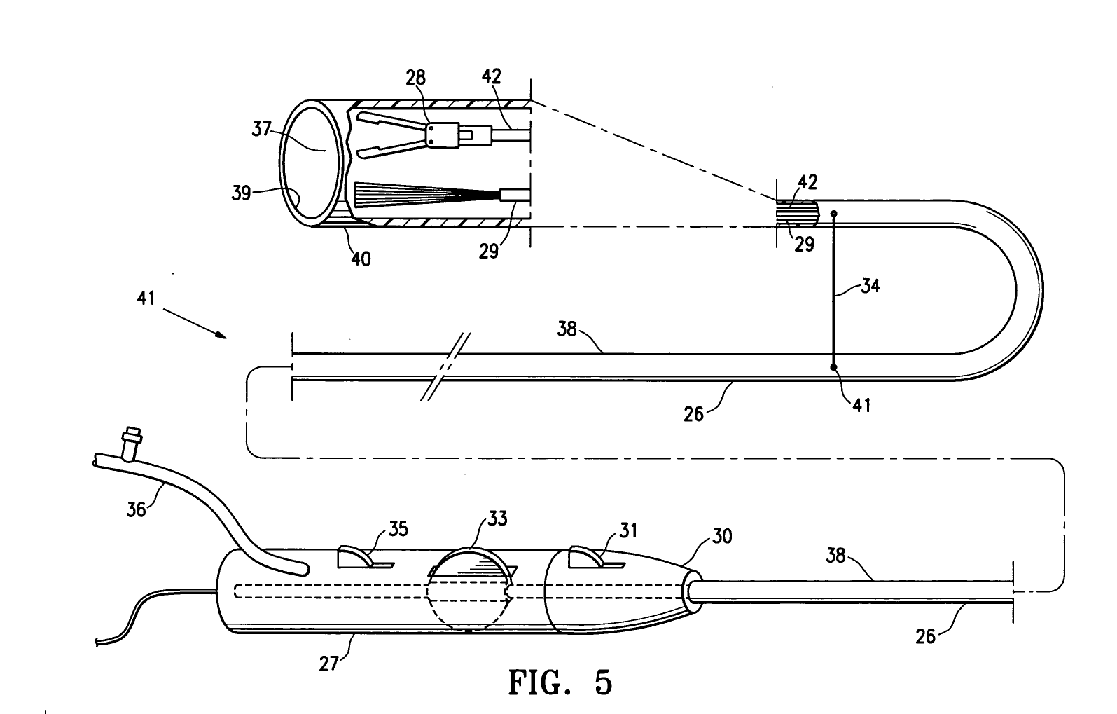

A treatment assembly 25 embodying features of the invention is shown in FIG. 5. The assembly 25 has a guide catheter 26 with an operative handle 27 on a proximal end of the catheter, a tissue grasping device 28 and a stabilizing member device 29 for positioning the free edges 21 and 22 of the patient's valve leaflets 14 and 15 in an appropriate position for grasping by the grasping device 28.

As shown in FIG. 5, the operative handle 27 of the assembly 25 is secured to the proximal end of guide catheter 26 and has a housing 30, a first control knob 31 for moving the grasping device 28 and delivery of the connecting clip 32, a rotating disc 33 for pulling deflecting wire 34 which controls the shape of the distal portion of the guide catheter 26 and a second control knob 35 for controlling the stabilizing device 29. A fluid conduit 36 is provided for delivery of contrast and other fluids through the handle 27 to the inner lumen 36 of the guide catheter 26.

Guide catheter 26 has an elongated shaft 38, an inner lumen 37 and a discharge port 39 in the distal end 40 which is in fluid communication with the inner lumen 37. A deflecting wire or strand 34 extends between the distal end 40 of the shaft and a location 41 proximal to the distal end. The proximal end of wire 34 is secured within the handle 27 so that by pulling on the wire 34, the distal portion of the shaft 38 can be shaped or otherwise deflected to a position orienting the discharge port 39 toward a desired location, e.g. the valve 13 to be treated.

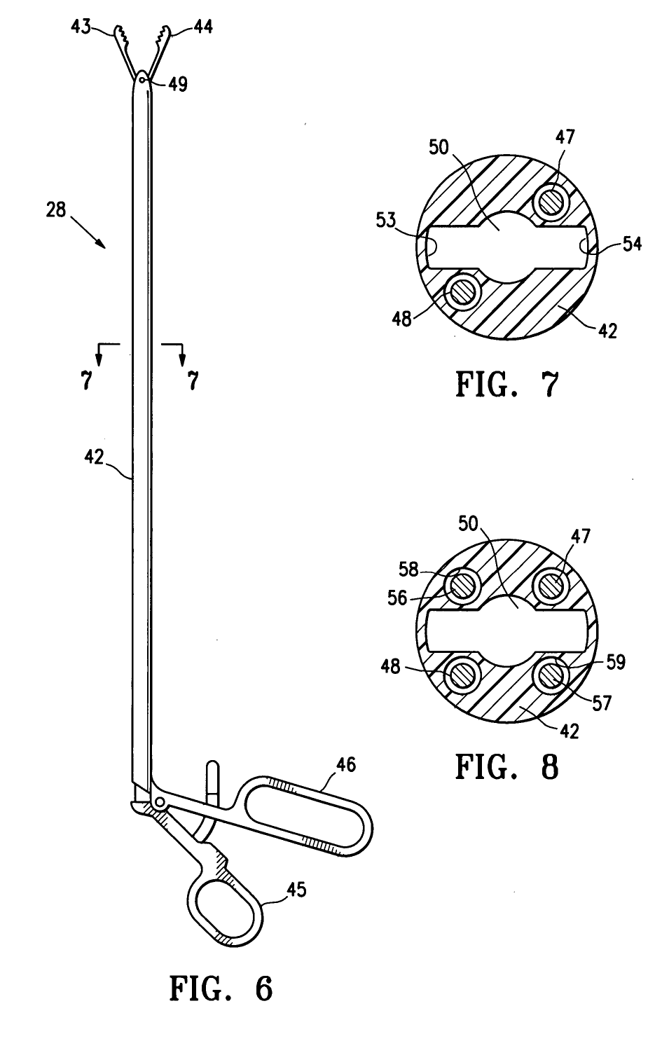

FIGS. 6-15 depict grasping device 28 which embodies features of the invention. The grasping device 28 has a flexible elongated shaft 42 suitable for percutaneous transvascular delivery to the patient's heart chamber, a plurality of grasping members or jaws 43 and 44 on the distal portion of the shaft and finger operated members 45 and 46 which operate the jaws through pull wires 47 and 48. The grasping members or jaws 44 and 45 are pivotally mounted at the pivot point 49 on the distal end of elongated shaft 42. While only two jaws 43 and 44 are shown, three or more jaws may be employed. The elongated shaft 42 of grasping device 28 has an inner lumen 50 extending therein to allow for the passage of instruments that aid or effect the deployment of a connecting member or leaflet clip 32 to the free edges 21 and 22 of the valve leaflets 14 and 15 to perform a Bow-Tie connection thereof as will be described in more detail hereinafter.

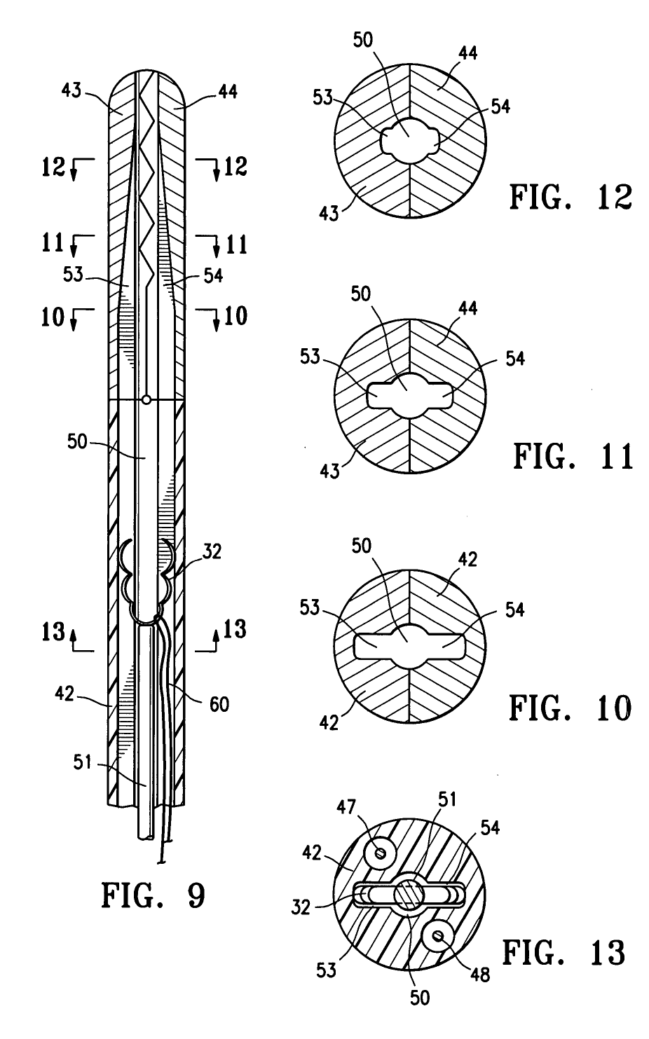

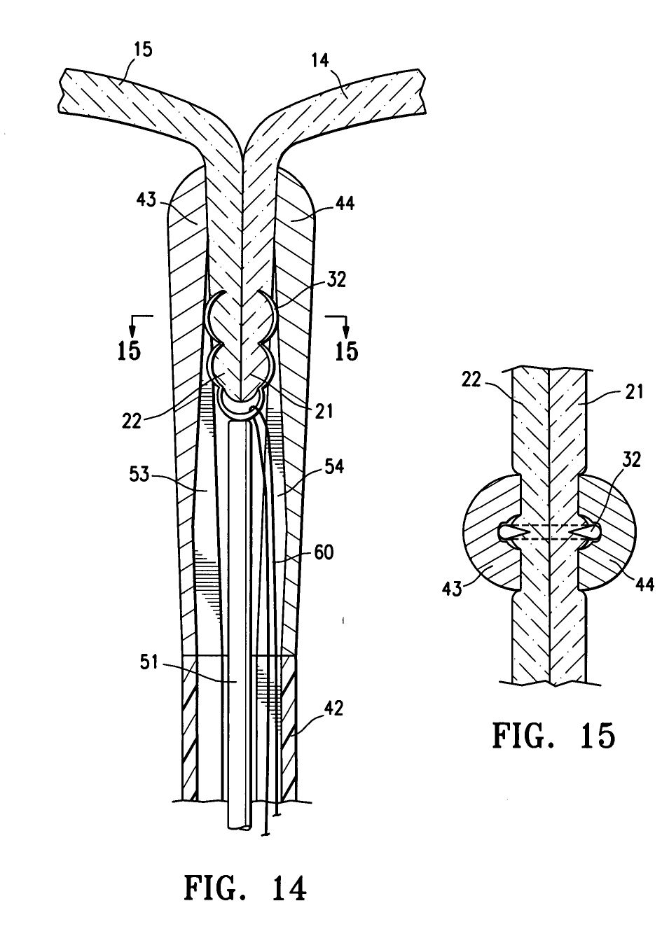

FIG. 9 is an enlarged elevational view in section of the distal portion of the grasping device 28 to illustrate delivery of leaflet clip 32 and the pusher bar 51 within lumen 50 which pushes the clip through the inner lumen of the grasping device. As shown in more detail in FIGS. 10-12, tapered grooves 53 and 54 are provided in the jaws 43 and 44 so that, as the clip 32 is pushed toward the distal ends of the jaws, the open distal ends of the clip slide along the tapered grooves and are closed against free edges 21 and 22 of the leaflets 14 and 15 grasped by the jaws. The deployed leaflet clip 32 closed against the free leaflet edges 21 and 22 in a Bow-Tie connection is shown in FIGS. 14 and 15. The inner lumen 50 continues through the jaws 42 and 43 to the ends of the jaws to allow passage of other instruments.

An alternative embodiment of grasping device 28 is shown in. FIG. 8 wherein wires or strands 56 and 57 are disposed in inner lumens 58 and 59 respectively and which have distal ends (not shown) secured at a distal location within the elongated shaft 42 to deflect or shape the distal portion of the shaft so as to position the jaws at a desired grasping location.

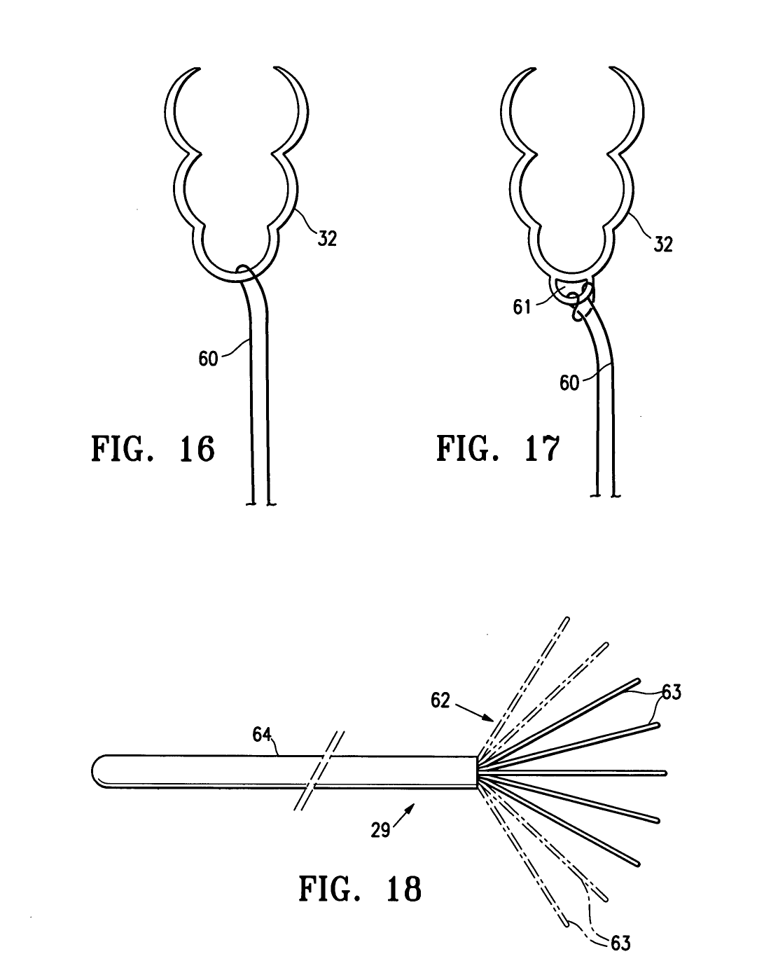

In FIG. 16, a clip 32 is shown with a strand 60 suitable to form an artificial cordae tendenae at the closed proximal end of the clip. An end of the strand 60 is secured to the closed proximal end of the clip 32 and another end secured to the ventricular wall in approximately the same orientation as the natural cordae tendenae. An alternate embodiment is depicted in FIG. 16B in which the closed proximal end of the clip 32 is provided with an eyelet 61. One end of the strand 60 is tied to the eyelet 61 and the other end to the heart wall as described above. A variety of clip structures may be employed to connect the free edges of the valve leaflets.

FIG. 18 illustrates the stabilizing device 29, which is slidably disposed within the inner lumen 37 of the guide catheter 26, has an expandable distal portion 62 having a plurality of arms or ribs 63 which are configured to engage the upstream side of the valve leaflets 14 and 15. The arms or ribs 63 are held together for delivery through the patient's vasculature and valve to be treated, but are expanded when the distal portion of the stabilizing device 29 is in an appropriate position to engage the atrial surfaces of the valve leaflets 14 and 15. The shaft 64 of the stabilizing device 29 is pulled proximally within the inner lumen 37 of guide catheter 26 to seat the arms or ribs 63 against the upstream leaflet surfaces and to position the free edges 21 and 22 of the leaflets 14 and 15 so that the jaws of the grasping device 28 can grasp the free edges. Preferably, the arms 63 are self-expanding so that they expand when they are deployed out of the inner lumen of the guide catheter. Alternatively, a strand or wire can be wrapped around the exterior of the expandable arms 61 for delivery and the strand or wire released when the arms exit the inner lumen of the guide catheter. Another alternative is to have an expanding mechanism enclosed within the arms in their contracted configuration which expands the arms once outside of the guide catheter. Connecting members may extend between the arms to control the expansion thereof. Additionally, a membrane may be employed over part of the expandable arm structure.

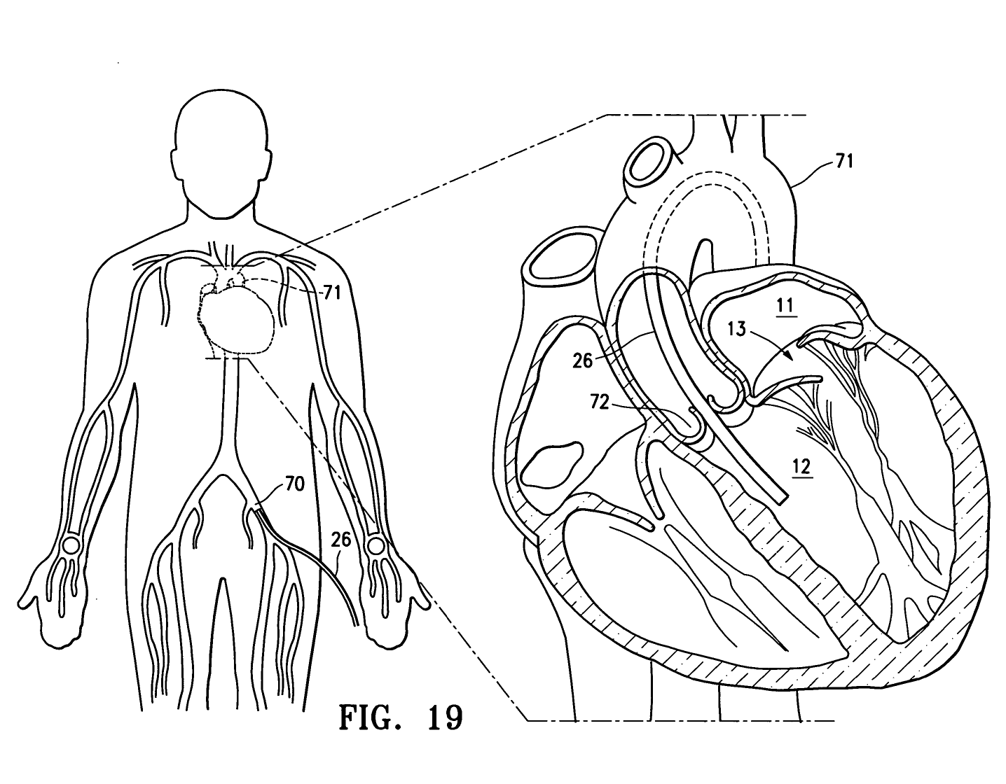

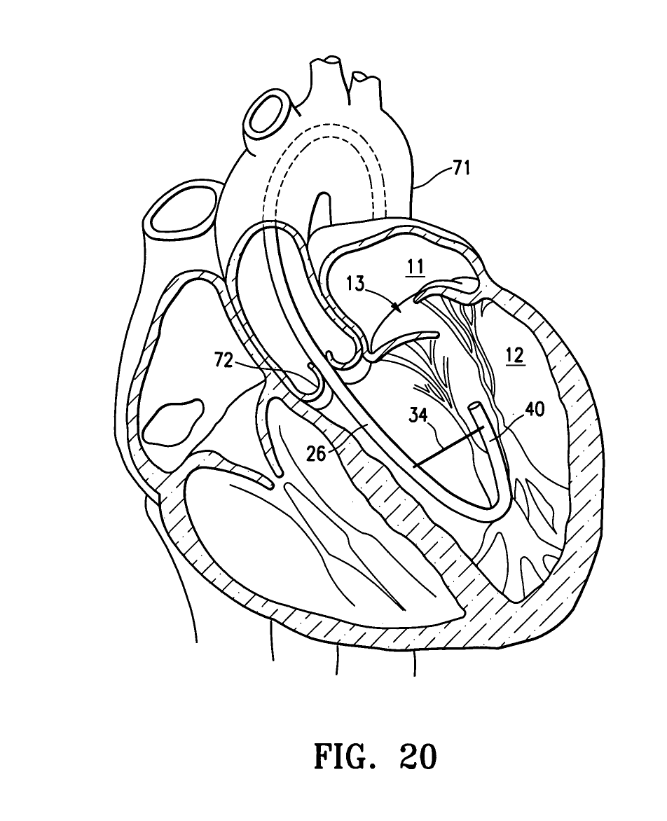

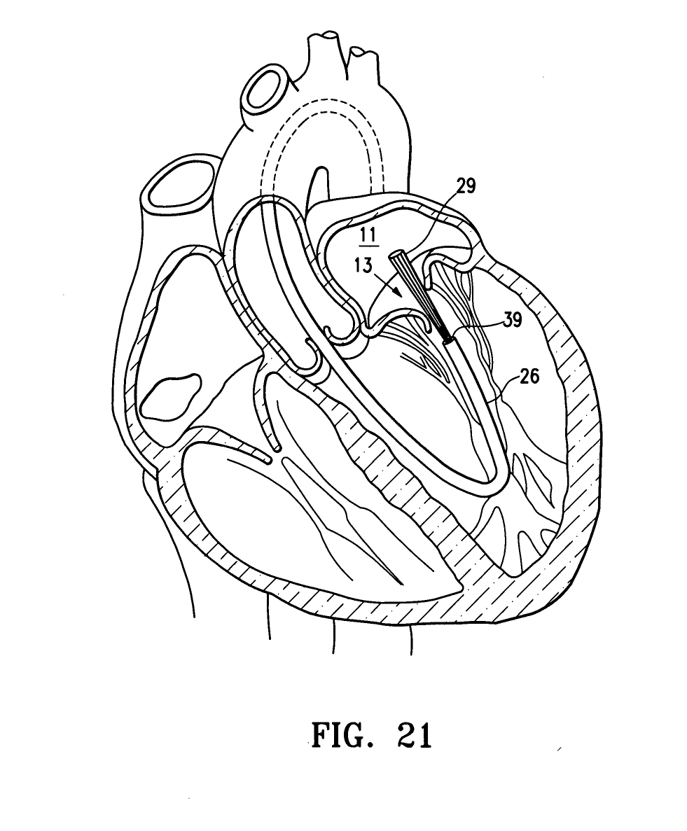

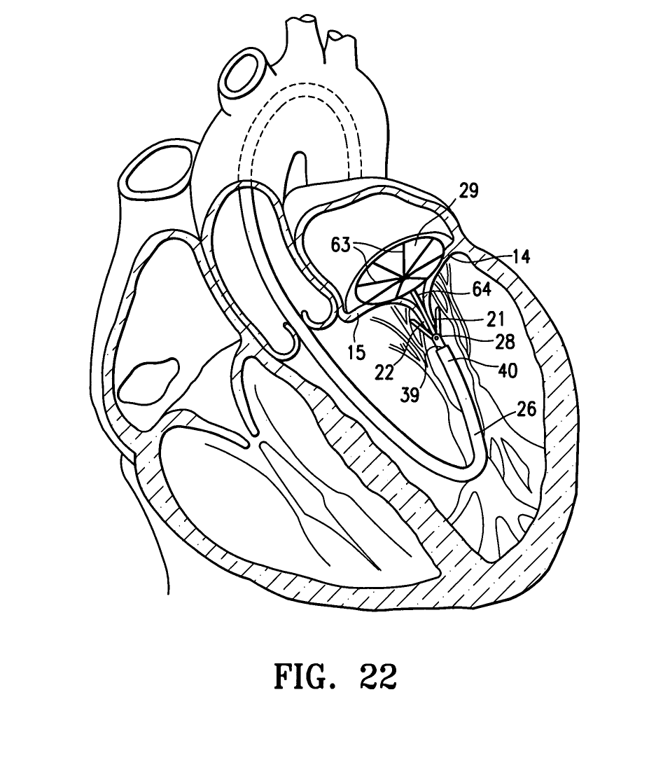

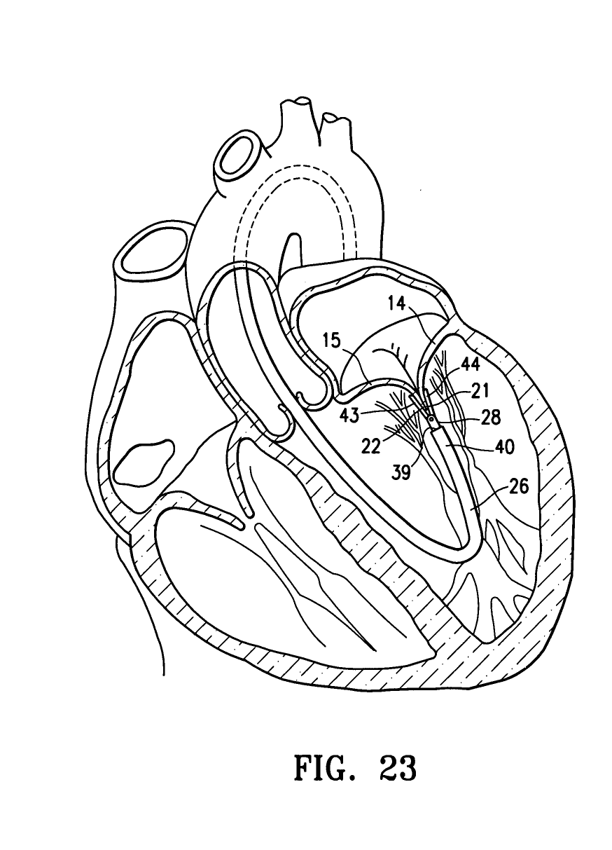

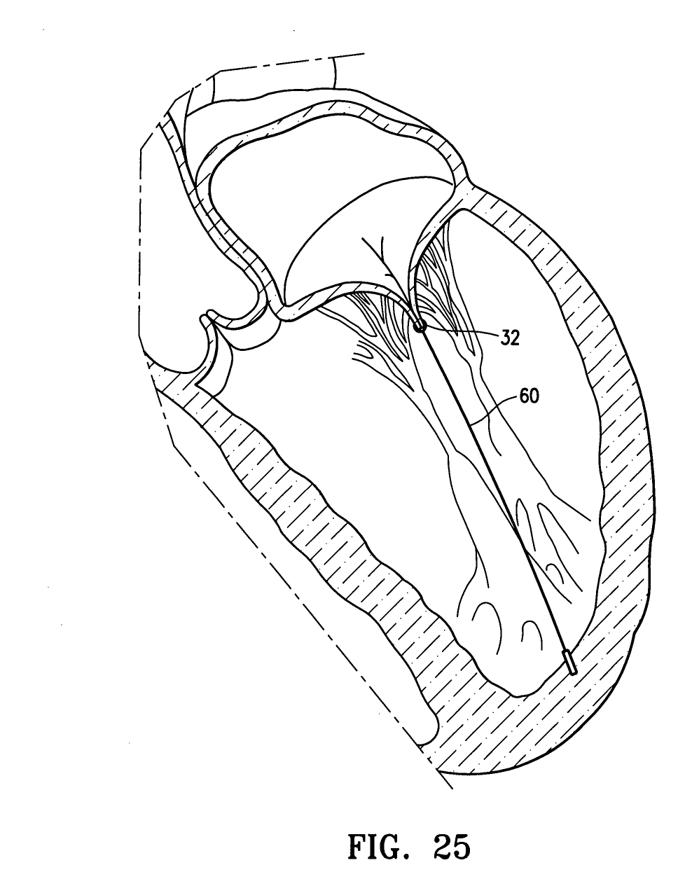

The use of the assembly 25 to make a Bow-Tie connection of the free edges 21 and 22 of the mitral valve 13 with an arterial approach is illustrated in FIGS. 19-26. As shown in FIG. 19, the guide catheter 26 is introduced into the patient's femoral artery 70 and properly advanced therein until the distal portion of the guide catheter has passed through the aorta 71 and aortic valve 72 and is deployed within the patient's left ventricle 12 with the distal portion of the guide catheter oriented toward the mitral valve 13 as shown in FIG. 20. The stabilizing device 29 is advanced out of the discharge port 39 in the guide catheter 26 through the mitral valve 13 into the left atrium 11 as shown in FIG. 21. The arms 63 are expanded within the left atrium 11 and the shaft 64 of the stabilizing device 29 is pulled proximally so the arms 63 engage the atrial surfaces of the valve leaflets 14 and 15 and put the free edges 21 and 22 into a grasping location as shown in FIG. 22. The grasping device 28 is advanced out of the discharge port 39 of guide catheter 26. The jaws 43 and 44 of the grasping device 28 may then be closed on the free edges 21 and 22 of valve leaflets 14 and 15 as shown in FIG. 23. The stabilizing device may then be withdrawn. As previously described, the leaflet clip 32 may then be advanced through the inner lumen 50 by pusher bar 51 to close the open ends of clip against and preferably into the grasped free edges 21 and 22 of valve leaflets 14 and 15 respectively, as shown in FIGS. 14 and 15. After the clip 32 has been deployed to form the Bow-Tie connection, the grasping device 28 and any other devices that may be present are withdrawn from the patient's heart chamber through inner lumen 36 of the guide catheter 31.

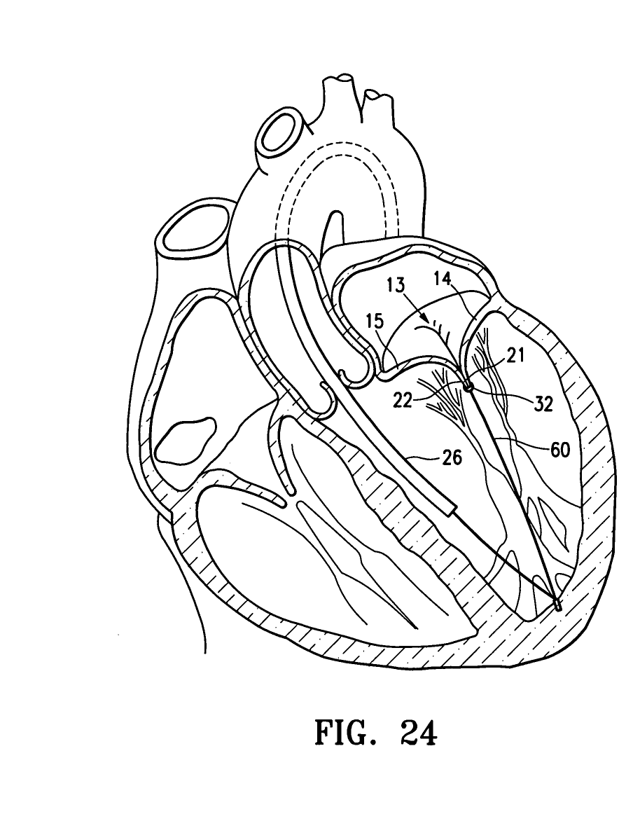

An embodiment is shown in FIG. 24 wherein an elongated strand 60 formed of relatively non-compliant material, such as PTFE, Nylon, polyethylene terephthalate, has its distal end secured to the closed proximal end of leaflet clip 32. The strand 60, if formed of PTFE, should have a transverse dimension of about 1 to about 3 mm. After deployment of the clip 32 to connect the free edges 21 and 22 of the leaflets 14 and 15 in a Bow-Tie connection, the proximal end of the strand 60 is pulled taut to position the leaflets 14 and 15 in a position to ensure proper closure during systole and then the proximal end of the strand 60 is secured to the ventricular wall. The proximal end of strand 60 may be sutured to the ventricle wall or may be secured thereto by a suitable anchor, hook or helical screw. This embodiment is particularly suitable in those instances wherein cordae tendenae connected to the valve leaflet are torn. The strand 60 acts as an artificial cordae tendenae to the leaflet. However, care must be exercised when securing the proximal end of the strand 60 is secured to the heart wall so that the valve leaflets are in a natural position in order to prevent or reduce regurgitation through the mitral valve 13.

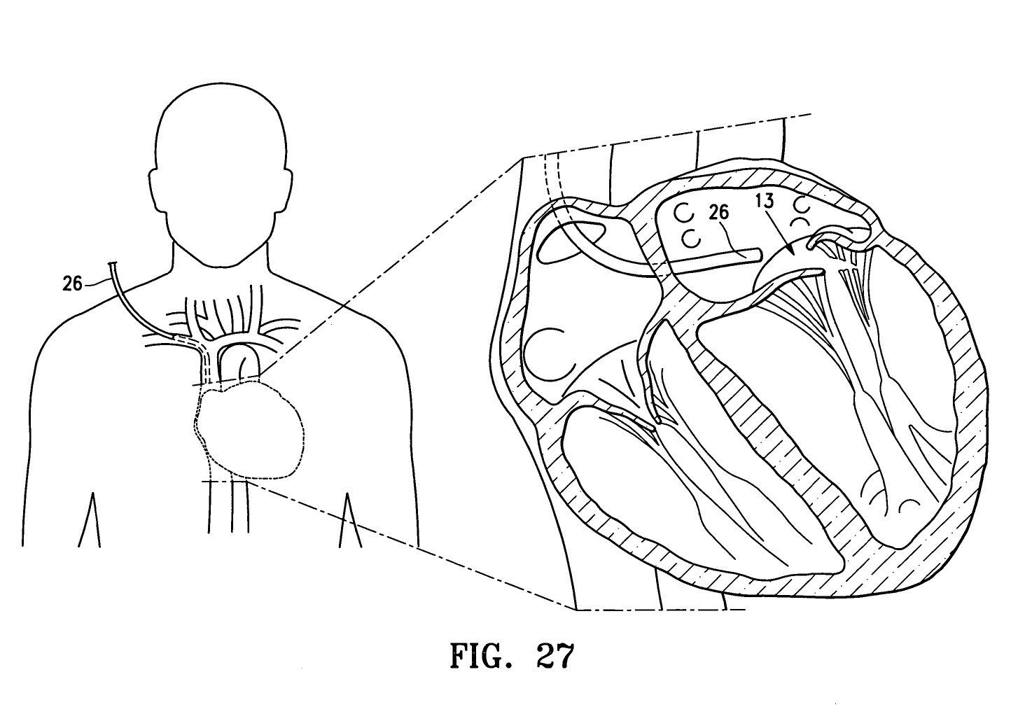

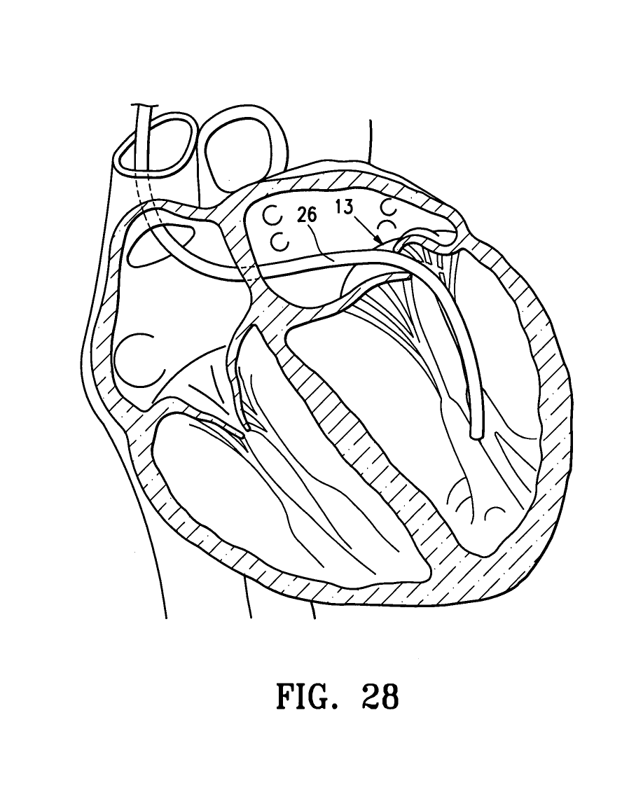

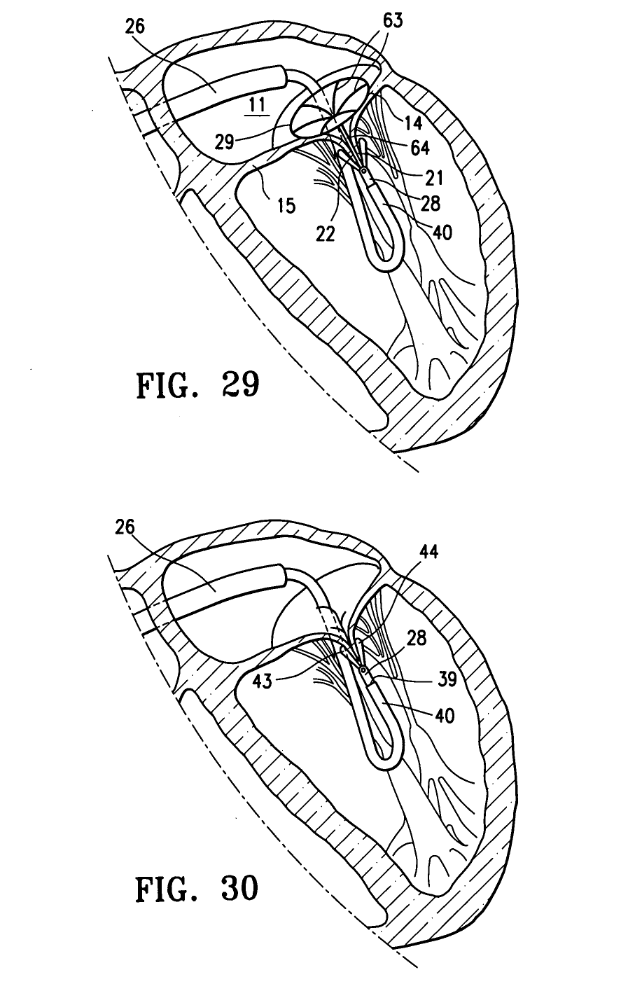

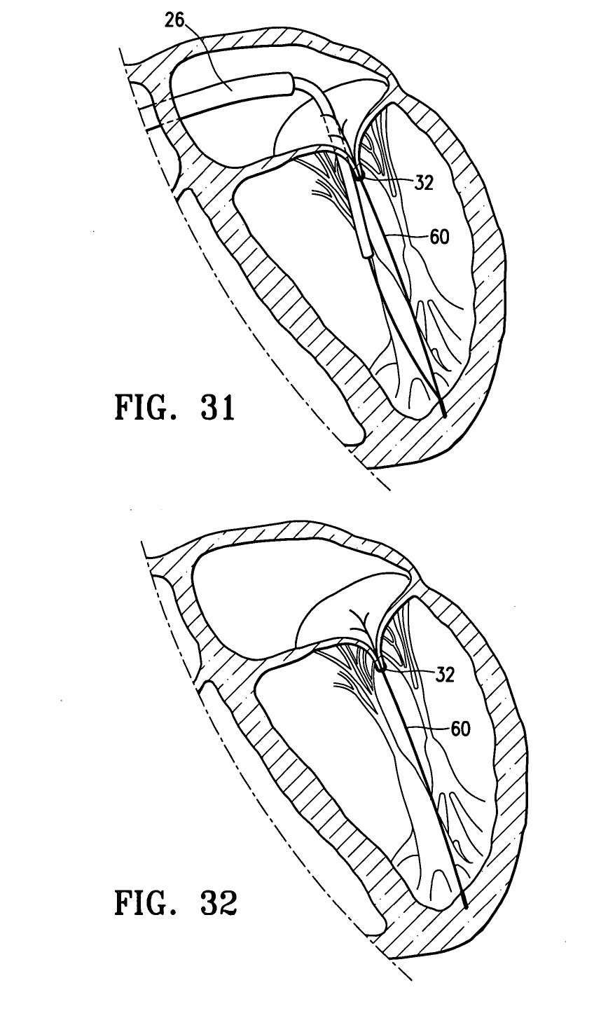

The use of the assembly 25 to make a Bow-Tie connection of the free edges 21 and 22 of the mitral valve 13 with an venous approach is illustrated in FIGS. 27-32. As shown in FIG. 27, the guide catheter 26 is introduced into the patient's right subclavical vein and properly advanced therein until the distal portion of the guide catheter has passed through the patient's superior vena cava and is deployed within the patient's right atrium. The distal portion of the guide catheter 26 is advanced through the patient's interatrial septum into the patient's left atrium. The distal portion of the guide catheter 26 is then advanced through the mitral valve 13 into the left ventricle where the distal portion of the guide catheter is oriented toward the mitral valve 13 as shown in FIG. 28. The distal portion of the guide catheter may be shaped by the deflecting wire 34 shown more clearly in FIG. 5. Other deflecting means may be employed. Alternatively, the guide catheter 26 may have an inner tubular member with a shaped distal tip that provided subselectivity such as shown in U.S. Pat. No. 6,251,104 (Kesten et al.). The stabilizing device 29 is advanced out of the discharge port 39 in the guide catheter 26 through the mitral valve 13 into the left atrium 11 as shown in FIG. 28. The arms 63 of the stabilizing device are expanded within the left atrium 11 and the shaft 62 of the stabilizing device 34 is pulled proximally so the arms engage the atrial surfaces of the valve leaflets 14 and 15 and put the free edges 21 and 22 into a grasping location as shown in FIG. 29. The grasping device 28 is advanced out of the discharge port 39 of guide catheter 26 and the jaws 43 and 44 of the grasping device 28 are then closed on the free edges 21 and 22 of valve leaflets 14 and 15 as shown in FIG. 30. The stabilizing device 29 may then be withdrawn. As previously described, the leaflet clip 32 may then be advanced through the inner lumen 50 by pusher bar 51 to close the open ends of clip 32 against and preferably into the grasped free edges 21 and 22 of valve leaflets 14 and 15 respectively, as shown in FIGS. 18 and 19. After the clip 32 has been deployed to form the Bow-Tie connection, the grasping device 28 and any other devices that may be present are withdrawn from the patient's heart chamber through inner lumen 37 of the guide catheter 26.

Alternative leaf stabilization devices such as inflatable balloons may be employed.

While particular forms of the invention have been illustrated and described, it will be apparent that various modifications can be made without departing from the spirit and scope of the invention. To the extent not otherwise described, the various components of the devices described herein may be formed of conventional materials and have conventional structures suitable for percutaneous transvascular delivery. Accordingly, it is not intended that the invention be limited to the specific embodiments illustrated. It is therefore intended that this invention to be defined by the scope of the appended claims as broadly as the prior art will permit. Moreover, those skilled in the art will recognize that features shown in one embodiment may be utilized in other embodiments. Terms such a “element”, “member”, “device”, “section”, “portion”, “component”, “means”, “steps” and words of similar import when used herein shall not be construed as invoking the provisions of 35 U.S.C. §112(6) unless the following claims expressly use the terms “means for” or “step for” followed by a particular function without specific structure or action. All patents and patent applications referred to above are hereby incorporated by reference in their entirety.

What is claimed is:

1. A percutaneous transvascular method of treating a patient' heart valve, comprising:
a. providing a guide catheter having an elongated shaft with a proximal end and a distal end, a discharge port in the distal end, an inner lumen extending therein to and in fluid communication with the discharge port and a distal portion configured or configurable to orient the discharge port toward the heart valve to be treated;
b. advancing the guide catheter through the patient's vascular system until the distal portion is disposed within a chamber of the patient's heart;
c. providing an elongated grasping device having a proximal end, an operable portion at the proximal end, a distal end, a pair of jaws on the distal end which can be opened and closed by the operable portion on the proximal end;
d. advancing a grasping device through the guide catheter until the pair of jaws extend out of the guide catheter;
e. providing a valve leaflet stabilizer having an expandable distal portion configured to engage one or more valve leaflets;
f. advancing an valve leaflet stabilizer member through the inner lumen of the guide catheter and through the valve to be treated;
g. expanding the expandable distal portion of the stabilizer member, engaging one or more leaflets of the valve and guiding one or more leaflets to a grasping location;
h. grasping one or more leaflets at the grasping location with the jaws of the grasping device; and
i. securing together free edges of one or more leaflets at the grasping location with at least one connecting member.

2. The method of claim 1 wherein the guide catheter includes a subselective tubular element with a shaped distal portion configured to extend out of the guide catheter to provide a desired orientation within the patient's heart chamber.

3. The method of claim 1 wherein the guide catheter has one or more deflecting strands to deflect the distal portion of the guide catheter to a desired orientation.

4. The method of claim 1 wherein an artificial cordae tendenae strand is provided and a first end of the artificial cordae tendenae strand is secured to at least one free edge of the valve leaflets and a second end of the artificial cordae tendenae strand is secured to a ventricular wall to hold the valve leaflet in a desired position.

5. The method of claim 3 wherein the artificial cordae tendenae strand is non-compliant.

6. The method of claim 5 wherein the artificial cordae tendenae strand is formed of a non-compliant material selected from the group consisting of nylon, polyethylene terephthalate and polytetrafluoroethylene.

7. The method of claim 1 wherein the grasping device has an inner lumen and the valve leaflet stabilizing member is passed through the inner lumen of the grasping device into the patient's heart.

8. The method of claim 1 wherein the opposed grasping jaws of the grasping device have inner grooves configured to receive a leaflet edge connecting member.

9. The method of claim 8 wherein the inner grooves of the grasping jaws taper distally to smaller dimensions to facilitate closing a leaflet edge connecting member when the connecting member is distally advanced therein.

10. A percutaneous system for treating a patient's heart having heart valve regurgitation, comprising:
a. guide catheter having an elongated shaft with a proximal end and a distal end, a discharge port in the distal end, an inner lumen extending therein to and in fluid communication with the discharge port and a shaped distal portion configured to orient the discharge port toward the heart valve to be treated;
b. an elongated grasping device which has a distal end, a pair of jaws on the distal end configured to grasp free ends of valve leaflets and which has a flexible shaft configured to be advanced through the guide catheter until the pair of jaws extend out of the guide catheter;
c. a stabilizing member having an expandable member on a distal portion thereof which is configured to be advanced through the guide catheter until the expandable member extends out of the guide catheter; and
d. a tissue connecting member to connect free edges of valve leaflets.

11. The minimally invasive system of claim 10 wherein the grasping jaws of the grasping device are configured to grasp the valve leaflets with free edges disposed together to facilitate securing the free edges together with the connecting member.

12. The minimally invasive system of claim 10 wherein the stabilizing member is configured to engage valve leaflets and hold the leaflets in a grasping location.

13. The minimally invasive system of claim 12 wherein the stabilizing member comprises a plurality of arms.

14. The system of claim 10 wherein the tissue connecting member has an artificial cordae tendenae strand secured thereto.

15. The system of claim 14 wherein the artificial cordae tendenae strand is configured to be taut during systole.

16. The system of claim 14 wherein the artificial cordae tendenae strand is configured to be flaccid during diastole.

17. The system of claim 14 wherein the artificial cordae tendenae strand is formed of relatively non-compliant polymeric material.
